(19) United States
(12) Patent Application Publication
(10) Pub. No.: US 2019/0192207 A1
(43) Pub. Date: Jun. 27, 2019

(54) FOOT CONTROL ASSEMBLY AND METHOD

(71) Applicant: Cryterion Medical, Inc., Carlsbad, CA (US)

(72) Inventor: Chadi Harmouche, Quebec (CA)

(21) Appl. No.: 16/226,033

(22) Filed: Dec. 19, 2018

Related U.S. Application Data
(60) Provisional application No. 62/608,916, filed on Dec. 21, 2017.

(51) Int. Cl. A61B 18/02; A61B 18/14

(52) U.S. Cl. CPC A61B 18/02; A61B 2018/00011; A61B 18/1492

(57) ABSTRACT

A foot control assembly for a user to control at least one stage of an ablation procedure. The ablation procedure can include an inflation stage, an ablation stage, a time to isolation and a thawing stage. The foot control assembly includes a controller and a plurality of spaced apart foot members. Each foot member is configured to be manually actuated. Each foot member sends at least one (i) initiation signal to the controller to initiate at least one stage of the ablation procedure, and/or (ii) termination signal to the controller to terminate at least one stage of the ablation procedure. For example, the initiation signal can initiate the inflation stage, the ablation stage and/or a calculation of time to isolation. The termination signal can terminate the inflation stage, the ablation stage and/or the thawing stage.

### CROSS-REFERENCE TO RELATED APPLICATION

This application claims priority to U.S. Provisional Application Ser. No. 62/608,916 filed on Dec. 21, 2017 and entitled “FOOT CONTROL ASSEMBLY AND METHOD”. As far as permitted, the contents of U.S. Provisional Application Ser. No. 62/608,916 are incorporated in their entirety herein by reference.

### TECHNICAL FIELD

The present disclosure relates to medical devices and methods for treating cardiac arrhythmias. More specifically, the disclosure relates to devices and methods for cardiac cryoablation.

### BACKGROUND

Cardiac arrhythmias involve an abnormality in the electrical conduction of the heart and are a leading cause of stroke, heart disease, and sudden cardiac death. Treatment options for patients with arrhythmias include medications, implantable devices, and catheter ablation of cardiac tissue.

Catheter ablation involves delivering ablative energy to tissue inside the heart to block aberrant electrical activity from depolarizing heart muscle cells out of synchrony with the heart's normal conduction pattern. The procedure is performed by positioning a portion of an energy delivery catheter adjacent to diseased or targeted tissue in the heart. The energy delivery component of the system is typically at or near a most distal (farthest from the user) portion of the catheter, and often at a tip of the device. Various forms of energy are used to ablate diseased heart tissue. These can include radio frequency (RF), ultrasound and laser energy, to name a few. One form of energy that is used to ablate diseased heart tissue includes cryogenics (also referred to herein as “cryoablation”). During an ablation procedure, with the aid of a guidewire, the distal tip of the catheter is positioned adjacent to diseased or targeted tissue, at which time the cryogenic energy can be delivered to create tissue necrosis, rendering the ablated tissue incapable of conducting electrical signals.

Atrial fibrillation is one of the most common arrhythmias treated using cryoablation. In the earliest stages of the disease, paroxysmal atrial fibrillation, the treatment strategy involves isolating the pulmonary veins from the left atrial chamber, a procedure that removes unusual electrical conductivity in the pulmonary vein. Recently, the use of techniques known as “balloon cryotherapy” catheter procedures to treat atrial fibrillation have increased. In part, this stems from ease of use, shorter procedure times and improved patient outcomes. During the balloon cryotherapy procedure, a refrigerant or cryogenic fluid (such as nitrous oxide, or any other suitable fluid) is delivered under pressure to an interior of one or more inflatable balloons which are positioned adjacent to or against the targeted cardiac tissue. Using this method, the extremely frigid cryogenic fluid causes necrosis of the targeted cardiac tissue, thereby rendering the ablated tissue incapable of conducting unwanted electrical signals.

Ablation procedures generally require the use of multiple hand-held and/or hand-controlled structures or devices. For example, a handle assembly may be handled or used by a user, an operator or another suitable health care physician or technician (hereinafter collectively referred to as “user”) to operate, position and/or control a catheter. Furthermore, a control console may often include various structures, components or devices, including a graphical display, which may require the user's manual control, guidance and/or input. There is thus a continuing need for improved cryoablation devices and systems.

### SUMMARY

The present disclosure is directed towards a foot control assembly for an intravascular catheter system. The foot control assembly is for a user to control at least one stage of an ablation procedure. The foot control assembly can include a controller and a plurality of spaced apart foot members. The plurality of foot members are each configured to be manually actuated by the user. Each foot member can send at least one initiation signal to the controller to initiate at least one stage of the ablation procedure, and/or termination signal to the controller to terminate at least one stage of the ablation procedure.

In various embodiments, the plurality of foot members can include a first foot member and a second foot member. In one embodiment, the first foot member and the second foot member can both be configured to be manually actuated by the user to send at least one initiation signal to the controller to initiate at least one stage of the ablation procedure. In another embodiment, the first foot member and the second foot member can each be configured to be manually actuated by the user to send at least one termination signal to the controller to terminate at least one stage of the ablation procedure. In still another embodiment, the first foot member can be configured to be manually actuated by the user to send at least one initiation signal to the controller to initiate at least one stage of the ablation procedure and the second foot member can be configured to be manually actuated by the user to send at least one termination signal to the controller to terminate at least one stage of the ablation procedure. Alternatively, the first foot member can be configured to be manually actuated by the user to send at least one termination signal to the controller to terminate at least one stage of the ablation procedure and the second foot member can be configured to be manually actuated by the user to send at least one initiation signal to the controller to initiate at least one stage of the ablation procedure.

In certain embodiments, at least one of the plurality of foot members can include at least one of a foot pedal, a button or a switch.

In certain embodiments, the ablation procedure can include an inflation stage. In one embodiment, the initiation signal can initiate the inflation stage. In another embodiment, the termination signal can terminate the inflation stage.

In some embodiments, the ablation procedure can include an ablation stage. In one embodiment, the initiation signal can initiate the ablation stage. In another embodiment, the termination signal can terminate the ablation stage.

In other embodiments, the ablation procedure can include a time to isolation. In one embodiment, the initiation signal can initiate a calculation of the time to isolation.

In still other embodiments, the ablation procedure can include a thawing stage. In one embodiment, the termination signal can terminate the ablation stage and can substantially simultaneously initiate the thawing stage. Alternatively, the termination signal can terminate the thawing stage.

In some embodiments, at least one foot member can be configured to be manually actuated by the user to send a timer signal to the controller to initiate a timer. In alternative embodiments, at least one foot member can be configured to be manually actuated by the user to send at least one of a deactivation signal to deactivate the foot control assembly, and/or an activation signal to activate the foot control assembly.

In various embodiments, each foot member is configured to be positioned on a support surface.

The present disclosure is also directed toward a method for controlling at least one stage of an ablation procedure. The method can include the step of sending with each of a plurality of foot members at least one initiation signal to the controller to initiate at least one stage of the ablation procedure, and/or termination signal to the controller to terminate at least one stage of the ablation procedure.

In one embodiment, the step of sending can include sending the initiation signal to the controller to initiate an inflation stage. In another embodiment, the step of sending can include sending the initiation signal to the controller to initiate an ablation stage. In still another embodiment, the step of sending can include sending the initiation signal to the controller to initiate a calculation of a time to isolation. In yet another embodiment, the step of sending can include sending the termination signal to the controller to terminate the inflation stage. In even another embodiment, the step of sending can include sending the termination signal to the controller to terminate the ablation stage. Alternatively, the step of sending can include sending the termination signal to the controller to terminate the ablation stage and substantially simultaneously initiate a thawing stage. Still alternatively, the step of sending can include sending the termination signal to the controller to terminate the thawing stage.

Further, the method can also include the step of sending with at least one foot member a timer signal to the controller to initiate a timer. Alternatively, the method can include the step of sending with at least one foot member a deactivation signal to the controller to deactivate the foot control assembly, and/or an activation signal to the controller to activate the foot control assembly.

Additionally, the present disclosure is also directed toward a foot control assembly for a user to control at least one stage of an ablation procedure. The foot control assembly can include a controller and a first foot member that is configured to be manually actuated by the user following a first foot member sequence. The first foot member sends at least a plurality of initiation signals to the controller to initiate at least one stage of the ablation procedure, and/or a plurality of termination signals to the controller to terminate at least one stage of the ablation procedure.

In one embodiment, the first foot member sequence can be predetermined by the user. In another embodiment, the first foot member sequence can be preprogrammed.

The ablation procedure includes an inflation stage, an ablation stage, a time to isolation and a thawing stage, as non-exclusive examples. In certain embodiments, the first foot member can be manually actuated a first time by the user to send a first initiation signal to the controller to initiate the inflation stage. In various embodiments, the first foot member can be manually actuated a plurality of times to send a second initiation signal to the controller to initiate the ablation stage. In some embodiments, the first foot member can be manually actuated the plurality of times to send a third initiation signal to the controller to initiate a calculation of the time to isolation. In other embodiments, the first foot member can be manually actuated the plurality of times to send a first termination signal to the controller to terminate the inflation stage. In still other embodiments, the first foot member can be manually actuated the plurality of times to send a second termination signal to the controller to terminate the ablation stage. In yet other embodiments, the first foot member can be manually actuated the plurality of times to send a third termination signal to the controller to terminate the thawing stage. Additionally, the first foot member can be manually actuated the plurality of times to send a timer signal to the controller to initiate a timer.

In some embodiments, the foot control assembly can include a second foot member that is configured to be manually actuated by the user following a second foot member sequence to send the timer signal to the controller to initiate the timer.

In other embodiments, the foot control assembly can include the second foot member that is configured to be manually actuated by the user following the second foot member sequence to send a deactivation signal to the controller to deactivate the foot control assembly, and/or an activation signal to the controller to activate the foot control assembly. For example, the second foot member can be actuated a first time to send the deactivation signal to the controller to deactivate the foot control assembly. Further, the second foot member can be actuated a plurality of times to send the activation signal to the controller to activate the foot control assembly.

In various embodiments, the foot control assembly can include the second foot member that is configured to be manually actuated by the user following the second foot member sequence to send the plurality of initiation signals to the controller to initiate at least one stage of the ablation procedure, and/or the plurality termination signals to the controller to terminate at least one stage of the ablation procedure. In one embodiment, the second foot member can be actuated a first time during the inflation stage to send the first termination signal to the controller to terminate the inflation stage. In another embodiment, the second foot member can be actuated a first time during the ablation stage to send the second termination signal to the controller to terminate the ablation stage. Alternatively, the second foot member can be actuated a first time during the ablation stage to send the second termination signal to the controller to terminate the ablation stage and substantially simultaneously initiate the thawing stage. In yet another embodiment, the second foot member can be actuated a first time during the thawing stage to send the third termination signal to the controller to terminate the thawing stage.

In certain embodiments, the first foot member is configured to be positioned on a support surface.

The present disclosure is further directed toward a method for controlling at least one stage of an ablation procedure. The method can include the step of manually actuating a first foot member following a first foot member sequence to send at least one of a plurality of initiation signals to the controller to initiate at least one stage of the ablation procedure, and/or a plurality of termination signals to the controller to terminate at least one stage of the ablation procedure.

In various embodiments, the step of manually actuating can include actuating the first foot member a first time to send a first initiation signal to the controller to initiate an inflation stage. In some embodiments, the step of manually actuating can include actuating the first foot member a plurality of times to send a second initiation signal to the controller to initiate an ablation stage. In other embodiments, the step of manual actuating can include actuating the first foot member the plurality of times to send a third initiation signal to the controller to calculate a time to isolation. In still other embodiments, the step of manual actuating can include actuating the first foot member the plurality of times to send a first termination signal to the controller to terminate the inflation stage. In yet other embodiments, the step of manual actuating can include actuating the first foot member the plurality of times to send a second termination signal to the controller to terminate the ablation stage. In even other embodiments, the step of manual actuating can include actuating the first foot member the plurality of times to send a third termination signal to the controller to terminate a thawing stage.

In some embodiments, the method can also include the step of manually actuating a second foot member following a second foot member sequence to send a timer signal to the controller to initiate a timer.

In other embodiments, the method can further include the step of manually actuating the second foot member following the second foot member sequence to send a deactivation signal to the controller to deactivate the foot control assembly, and/or an activation signal to the controller to activate the foot control assembly. In one embodiment, the step of manually actuating can include actuating the second foot member a first time to send the deactivation signal to the controller to deactivate the foot control assembly. In another embodiment, the step of manually actuating can include actuating the second foot member a plurality of times to send the activation signal to the controller to activate the foot control assembly.

In certain embodiments, the method can further include the step of manually actuating the second foot member following the second foot member sequence to send the plurality of initiation signals to the controller to initiate at least one stage of the ablation procedure, and/or the plurality of termination signals to the controller to terminate at least one stage of the ablation procedure. In one embodiment, the step of manually actuating can include actuating the second foot member a first time during the inflation stage to send the first termination signal to the controller to terminate the inflation stage. In another embodiment, the step of manually actuating can include actuating the second foot member a first time during the ablation stage to send the second termination signal to the controller to terminate the ablation stage. Alternatively, the step of manually actuating can include actuating the second foot member a first time during the ablation stage to send the second termination signal to the controller to terminate the ablation stage and substantially simultaneously initiate the thawing stage. In still another embodiment, the step of manually actuating can include actuating the second foot member a first time during the thawing stage to send the third termination signal to the controller to terminate the thawing stage.

Additionally, the method can also include the step of manually actuating a third foot member following a third foot member sequence to send the timer signal to the controller to initiate the timer.

Further, in some applications, the present disclosure is directed toward a foot control assembly for a user to control a flow rate of a cryogenic fluid to a balloon catheter. The foot control assembly can include a controller and a first foot member that is configured to be manually actuated by the user. The first foot member sends a first depression signal to the controller to control the flow rate of the cryogenic fluid to the balloon catheter when the first foot member is depressed and held down. The first foot member sends a first release signal to the controller to maintain the flow rate of the cryogenic fluid to the balloon catheter when the first foot member is released.

In various embodiments, the first depression signal can be sent to the controller each time the first foot member is depressed and held down. Further, the first release signal can be sent to the controller each time the first foot member is released.

In some embodiments, the foot control assembly can further include a second foot member that is configured to be manually actuated by the user. The second foot member sends a second depression signal to the controller to control the flow rate of the cryogenic fluid to the balloon catheter when the second foot member is depressed and held down. The second foot member sends a second release signal to the controller to maintain the flow rate of the cryogenic fluid to the balloon catheter when the second foot member is released.

In certain embodiments, the first foot member and the second foot member are configured to be positioned on a support surface.

The present disclosure is also directed toward a method for controlling a flow rate of a cryogenic fluid to a balloon catheter. The method can include the step of manually actuating a first foot member to send at least one first depression signal to the controller to control the flow rate of the cryogenic fluid to the balloon catheter, and/or first release signal to the controller to maintain the flow rate of the cryogenic fluid to the balloon catheter.

In certain embodiments, the step of manually actuating can include depressing and holding down the first foot member and/or releasing the first foot member.

In some embodiments, the method can also include the step of manually actuating a second foot member to send at least one second depression signal to the controller to control the flow rate of the cryogenic fluid to the balloon catheter, and/or second release signal to the controller to maintain the flow rate of the cryogenic fluid to the balloon catheter.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a schematic side view of a patient, a user and an embodiment of an intravascular catheter system having features of the present disclosure, including one embodiment of a foot control assembly;

FIG. 2 is a schematic side view of the patient, the user and another embodiment of the intravascular catheter system, including another embodiment of the foot control assembly; and

FIG. 3 is a flowchart illustrating one embodiment of a method for operating the foot control assembly.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

Embodiments of the present disclosure are described herein in the context of a foot control assembly for an intravascular catheter system. Those of ordinary skill in the art will realize that the following detailed description of the present disclosure is illustrative only and is not intended to be in any way limiting. Other embodiments of the present disclosure will readily suggest themselves to such skilled persons having the benefit of this disclosure. Reference will now be made in detail to implementations of the present disclosure as illustrated in the accompanying drawings.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application-related and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

Although the disclosure provided herein focuses mainly on cryogenics, it is understood that various other forms of ablative energy can be used to ablate diseased heart tissue. These can include radio frequency (RF), ultrasound, pulsed DC electric fields and laser energy, as non-exclusive examples. The present disclosure is intended to be effective with any or all of these and other forms of energy.

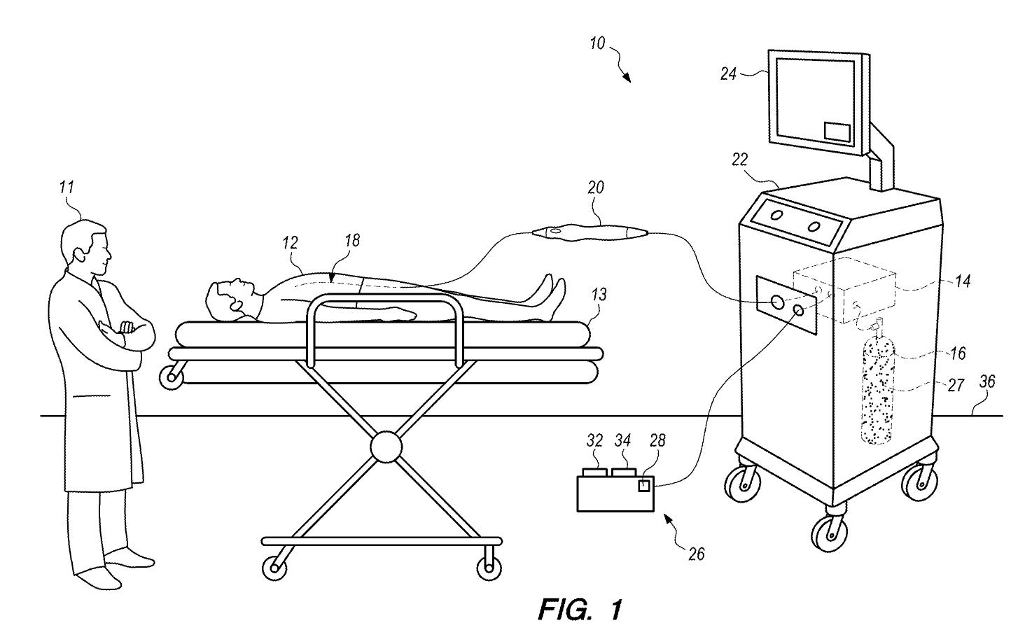

FIG. 1 is a side view illustration of one embodiment of an intravascular catheter system 10 (also sometimes referred to herein as a “catheter system”) for use by a user 11, such as a health care professional, with a patient 12, which can be a human being or an animal. In this embodiment, the user 11 operates and/or controls the catheter system 10 to perform the ablation procedure on the patient 12. While FIG. 1 shows only one user 11, it is understood that a plurality of different users 11 can operate or assist in the operation of the catheter system 10 at the same or at different times throughout the ablation procedure. In other words, the user 11 illustrated in FIG. 1 can represent any number of different users 11, i.e., a first user, a second user, etc. Further, it is understood that while specific reference is made to the user 11 as a healthcare professional, healthcare professional can include an operator, a physician, a physician's assistant, nurse and/or any other suitable person and/or individual.

In the embodiment illustrated in FIG. 1, the patient 12 is positioned on a gurney 13. However, it is understood that the patient 12 can be positioned on any suitable surface, such as a table or a bed, as non-exclusive examples.

Although the catheter system 10 is specifically described herein with respect to the intravascular catheter system, it is understood and appreciated that other types of catheter systems and/or ablation systems can equally benefit by the teachings provided herein. For example, in certain non-exclusive alternative embodiments, the present disclosure can be equally applicable for use with any suitable types of ablation systems and/or any suitable types of catheter systems. Thus, the specific reference herein to use as part of the intravascular catheter system is not intended to be limiting in any manner.

The design of the catheter system 10 can be varied. In certain embodiments, such as the embodiment illustrated in FIG. 1, the catheter system 10 can include one or more of a control system 14, a fluid source 16 (e.g., one or more fluid containers), a balloon catheter 18, a handle assembly 20, a control console 22, a graphical display 24 (also sometimes referred to as a graphical user interface or “GUI”) and a foot control assembly 26. It is understood that although FIG. 1 illustrates the structures of the catheter system 10 in a particular position, sequence and/or order, these structures can be located in any suitably different position, sequence and/or order than that illustrated in FIG. 1. It is also understood that the catheter system 10 can include fewer or additional structures than those specifically illustrated and described herein.

In various embodiments, the control system 14 is configured to monitor and control the various processes of an ablation procedure. More specifically, the control system 14 can monitor and control release and/or retrieval of a cryogenic fluid 27 to and/or from the balloon catheter 18. The control system 14 can also control various structures that are responsible for maintaining or adjusting a flow rate and/or a pressure of the cryogenic fluid 27 that is released to the balloon catheter 18 during the ablation procedure. In various embodiments, the catheter system 10 delivers ablative energy in the form of the cryogenic fluid 27 to cardiac tissue of the patient 12 to create tissue necrosis, rendering the ablated tissue incapable of conducting electrical signals. Additionally, in various embodiments, the control system 14 can control activation and/or deactivation of one or more other processes of the balloon catheter 18. Additionally, or in the alternative, the control system 14 can receive electronic signals, data and/or other information (also sometimes referred to as “sensor output”) from various structures within the catheter system 10. In certain embodiments, the control system 14 and/or the GUI 24 can be electrically connected and/or coupled. In some embodiments, the control system 14 can receive, monitor, assimilate and/or integrate any sensor output and/or any other data or information received from any structure within the catheter system 10 in order to control the operation of the balloon catheter 18. Still further, or in the alternative, the control system 14 can control positioning of portions of the balloon catheter 18 within a circulatory system (also sometimes referred to herein as the “body”) of the patient 12, and/or can control any other suitable functions of the balloon catheter 18.

The fluid source 16 (also sometimes referred to as “fluid container 16”) can include one or more fluid container(s) 16. It is understood that while one fluid container 16 is illustrated in FIG. 1, any suitable number of fluid containers 16 may be used. The fluid container(s) 16 can be of any suitable size, shape and/or design. The fluid container(s) 16 contains the cryogenic fluid 27, which is delivered to an ablation element (e.g., a balloon) on the balloon catheter 18 with or without input from the control system 14 during the ablation procedure. Once the ablation procedure has initiated, the cryogenic fluid 27 can be injected or delivered and the resulting gas, after a phase change, can be retrieved from the balloon catheter 18, and can either be vented or otherwise discarded as exhaust. More specifically, the cryogenic fluid 27 delivered to and/or removed from the balloon catheter 18 can include a flow rate that varies. Additionally, the type of cryogenic fluid 27 that is used during the ablation procedure can vary. In one non-exclusive embodiment, the cryogenic fluid 27 can include liquid nitrous oxide. In another non-exclusive embodiment, the cryogenic fluid 27 can include liquid nitrogen. However, any other suitable cryogenic fluid 27 can be used.

The design of the balloon catheter 18 can be varied to suit the design requirements of the catheter system 10. As shown, the balloon catheter 18 is inserted into the body of the patient 12 during the ablation procedure. In one embodiment, the balloon catheter 18 can be positioned within the body of the patient 12 using the control system 14. Stated in another manner, the control system 14 can control positioning of the balloon catheter 18 within the body of the patient 12. Alternatively, the balloon catheter 18 can be manually positioned within the body of the patient 12 by the user 11. In certain embodiments, the balloon catheter 18 is positioned within the body of the patient 12 utilizing at least a portion of the sensor output that is received from the balloon catheter 18. For example, in various embodiments, the sensor output is received by the control system 14, which can then provide the user 11 with information regarding the positioning of the balloon catheter 18. Based at least partially on the sensor output feedback received by the control system 14, the user 11 can adjust the positioning of the balloon catheter 18 within the body of the patient 12 to ensure that the balloon catheter 18 is properly positioned relative to targeted cardiac tissue. While specific reference is made herein to the balloon catheter 18, as noted above, it is understood that any suitable type of medical device and/or catheter may be used.

The handle assembly 20 is handled and used by the user 11 to operate, position and/or control the balloon catheter 18. The design and specific features of the handle assembly 20 can vary to suit the design requirements of the catheter system 10. In the embodiment illustrated in FIG. 1, the handle assembly 20 is separate from, but in electrical and/or fluid communication with the control system 14, the fluid container 16 and the GUI 24. In some embodiments, the handle assembly 20 can integrate and/or include at least a portion of the control system 14 within an interior of the handle assembly 20. In one embodiment, the user 11 can steer and/or navigate the balloon catheter 18 by utilizing the handle assembly 20. It is understood that the handle assembly 20 can include fewer or additional components than those specifically illustrated and described herein.

In the embodiment illustrated in FIG. 1, the control console 22 includes at least a portion of the control system 14, the fluid container 16 and/or the GUI 24. However, in alternative embodiments, the control console 22 can contain additional structures not shown or described herein. Still alternatively, the control console 22 may not include various structures that are illustrated within the control console 22 in FIG. 1. For example, in certain non-exclusive alternative embodiments, the control console 22 does not include the GUI 24.

In various embodiments, the GUI 24 is electrically connected to the control system 14. Additionally, the GUI 24 provides the user 11 of the catheter system 10 with information that can be used before, during and/or after the ablation procedure. For example, the GUI 24 can provide the user 11 with information based on the sensor output, and any other relevant information that can be used before, during and/or after the ablation procedure. The specifics of the GUI 24 can vary depending upon the design requirements of the catheter system 10, or the specific needs, specifications and/or desires of the user 11.

In one embodiment, the GUI 24 can provide static visual data and/or information to the user 11. In addition, or in the alternative, the GUI 24 can provide dynamic visual data and/or information to the user 11, such as video data or any other data that changes over time, e.g., during the ablation procedure. Further, in various embodiments, the GUI 24 can include one or more colors, different sizes, varying brightness, etc., that may act as alerts to the user 11. Additionally, or in the alternative, the GUI 24 can provide audio data or information to the user 11.

As an overview, and as provided in greater detail herein, the foot control assembly 26 allows the user 11 to manually operate and/or control certain stages of the ablation procedure. As used herein, the term “manually” can include the user 11 using his or her foot or feet, in contrast to his or her hand(s), to operate and/or control at least a portion and/or a stage of the ablation procedure. As used herein, foot or feet can include any portion of the leg or lower extremities of the user 11, including any attachment thereto, such as a shoe, for example. Furthermore, as described in greater detail, each ablation procedure can include one or more stages, such as: (i) an inflation stage, (ii) an ablation stage, (iii) a time to isolation, and/or (iv) a thawing stage, as non-exclusive examples. Alternatively, the ablation procedure may also include other stages not specifically mentioned herein.

As utilized herein, the “inflation stage” refers generally to the portion of the ablation procedure, wherein the cryogenic fluid 27 is being delivered from the fluid source 16 to the balloon catheter 18 at a flow rate that does not cause tissue necrosis. More specifically, the cryogenic fluid 27 is being delivered to the inflatable balloon of the balloon catheter 18. During the inflation stage, the user 11 may adjust and/or position the balloon catheter 18 within the body of the patient 12 to achieve positioning of the inflatable balloon adjacent to a targeted tissue of the patient 12. The targeted tissue can include at least a portion of heart tissue of the patient 12 that is to be treated by the catheter system 10, such as an ostium of a pulmonary vein, for example. Once positioned adjacent to the targeted tissue and the pulmonary vein is occluded, ablation of the targeted tissue may be initiated.

The “ablation stage” refers generally to the cryogenic fluid 27 being delivered from the fluid source 16 to the inflatable balloon of the balloon catheter 18 at a flow rate to create tissue necrosis. Tissue necrosis has the effect of rendering targeted tissue incapable of conducting cardiac electrical signals. During ablation of the targeted tissue, the inflatable balloon of the balloon catheter 18 is positioned adjacent to targeted tissue, with the pulmonary vein being occluded.

The “time to isolation” or “time to effect” refers to the moment when cardiac electrical signals during the ablation procedure are lost or “isolated” due to tissue ablation. It is appreciated that the time to isolation is a variable that is determined only through the process of the ablation procedure, and potentially may not actually be achieved in any given ablation procedure. As such, although the ablation procedure can be said to include a time to isolation, it is understood that the specific time to isolation for any given ablation procedure is actually unknown and only a potentiality until it happens (if it does at all) during the ablation procedure. One representative example of time to isolation would be when signals from a left atrium no longer appear in the pulmonary vein due to a circumferential lesion.

Additionally, the “thawing stage” refers generally to the stage of the ablation procedure, wherein targeted tissue of the patient 12 that has been ablated is allowed to thaw to a certain temperature and/or for a certain period of time. The thawing stage can be temperature based, time based, or both. Temperature based means that the ablated heart tissue is allowed to thaw to a certain temperature. Time based means the ablated heart tissue is allowed to thaw for a certain period of time. The temperature and period of time can vary depending on the patient 12 and/or any other cryoablation parameters. During the thawing stage of the targeted tissue of the patient 12, the cryogenic fluid 27 may be delivered from the fluid source 16 to the inflatable balloon of the balloon catheter 18 and/or retrieved from the inflatable balloon of the balloon catheter 18, but at a flow rate sufficient to maintain the inflatable balloon at least partially or substantially inflated to prevent the balloon catheter 18 from falling out of position and/or to reduce the likelihood of tissue damage to the patient 12.

In certain embodiments, the foot control assembly 26 can be used to initiate and/or terminate any stage of the ablation procedure. As non-exclusive examples, the foot control assembly 26 can be used to initiate and/or terminate the inflation stage, the ablation stage and/or the thawing stage. In other embodiments, the foot control assembly 26 can allow the user 11 to time, measure and/or calculate different events and/or stages of the ablation procedure, such as time to isolation. In yet other embodiments, the foot control assembly 26 can initiate and/or terminate timers and/or other predetermined events. Additionally, and/or alternatively, the foot control assembly 26 can perform any other suitable function of the catheter system 10 that may be manually controlled by the user 11.

The design and specific features of the foot control assembly 26 can vary to suit the design requirements of the catheter system 10. In the embodiment illustrated in FIG. 1, the foot control assembly 26 can include one or more of a controller 28 and a plurality of foot members, i.e., a first foot member 32, a second foot member 34, a third foot member (not shown), etc. The plurality of foot members can be spaced apart from one another. It is recognized that the terms “first foot member 32,” “second foot member 34,” “third foot member,” etc. can be used interchangeably. In this embodiment, while specific reference is made herein to the first foot member 32 and the second foot member 34, it is further recognized that the foot control assembly 26 can include any number of foot members, which may allow the user 11 to manually control any suitable function of the catheter system 10. Further, it is understood that the foot control assembly 26 can include fewer or additional components than those specifically illustrated and described herein.

In the embodiment illustrated in FIG. 1, the foot control assembly 26 is designed as a single structure that is coupled and/or connected to the control system 14. The foot control assembly 26 can be electrically and/or mechanically coupled and/or connected to the control system 14 via any suitable manner. Alternatively, the foot control assembly 26 can be coupled and/or connected to other structures of the catheter system 10. Additionally, and/or in the alternative, the foot control assembly 26, can be designed to include various structures.

Additionally, as illustrated in FIG. 1, the foot control assembly 26 can be positioned or otherwise situated on or near a support surface 36, such as a floor, for example. In the embodiment illustrated in FIG. 1, the user 11, the gurney 13 and the control console 22 can also be positioned or otherwise situated on or near the support surface 36. The foot control assembly 26 can be positioned on or near the support surface via any suitable manner. In one embodiment, the foot control assembly 26 can include wheels. Alternatively, the foot control assembly 26 can rest directly on the support surface 36, or the foot control assembly 26 can be positioned on top of a non-skid pad or some other dampening material, for example.

In various embodiments, the controller 28 is configured to receive and/or process electronic or other signals. In certain embodiments, the controller 28 can receive and/or process signals to initiate and/or terminate varying stages of the ablation procedure. More specifically, the controller 28 can receive and/or process signals to initiate and/or terminate the inflation stage, the ablation stage and/or the thawing stage, as non-exclusive examples. In alternative embodiments, the controller 28 can receive and/or process signals to time, measure and/or calculate different stages of the ablation procedure. For example, the controller 28 can calculate and/or measure the time to isolation. Additionally, the controller 28 can receive and/or process other signals to perform any other suitable function.

In the embodiment illustrated in FIG. 1, the controller 28 can be integrated and/or included as part of the foot control assembly 26. In other embodiments, the controller 28 can be positioned remotely from the foot control assembly 26. For example, the controller 28 can be integrated and/or included as part of the control system 14 and/or control console 22.

In various embodiments, the controller 28 can include at least one processor (e.g., microprocessor) that executes software and/or firmware stored in memory of the controller 28. The software/firmware code contains instructions that, when executed by the processor, cause the controller 28 to perform the functions of the control algorithm described herein. The controller 28 may alternatively include one or more application-specific integrated circuits (ASICs), field-programmable gate arrays (FPGAs), digital signal processors (DSPs), hardwired logic, or combinations thereof. The controller 28 may receive information from a plurality of system 10 components and feed the information (e.g., sensor data, signals from the foot control assembly 26, and user inputs from the GUI 24) into a control algorithm which determines at least one control parameter which may in part govern operation of the catheter system 10.

In certain embodiments, the first foot member 32 can be selectively and/or manually actuated by the user 11 to send a plurality of initiation signals to initiate one or more stages of the ablation procedure. As one non-exclusive example, the first foot member 32 can be selectively and/or manually actuated by the user 11 to send a first initiation signal to the controller 28. In this embodiment, while specific reference is made herein to sending the first initiation signal, it is recognized that the first foot member 32 can send one or more initiation signals, i.e., the first initiation signal, a second initiation signal, a third initiation signal, etc. to initiate certain stages of the ablation procedure, which can be collectively referred to herein as an “initiation signal.” It is understood that first initiation signal, the second initiation signal, the third initiation signal, etc. can be used interchangeably. In various embodiments, the controller 28 can process the first initiation signal and can initiate one or more stages of the ablation procedure. Furthermore, the first foot member 32 can have any suitable design that can enable the user 11 to selectively and/or manually actuate the first foot member 32.

In various embodiments, the first foot member 32 can send one or more initiation signals to the controller 28 to initiate certain stages of the ablation procedure depending on how the user 11 actuates the first foot member 32. More specifically, the function or operation of the first foot member 32 can depend on how the user 11 actuates the first foot member 32. In other words, the function or operation of the first foot member 32 can depend on a first foot member sequence. As referred to herein, the term “first foot member sequence” can include the method or manner in which the first foot member 32 is actuated, i.e., a number of times, an order, an arrangement, a series, a length of time, etc. and/or any combination thereof. In this embodiment, while specific reference is made to the first foot member sequence, it is recognized that the foot control assembly 26 can include any number of foot member sequences, i.e., the first foot member sequence, a second foot member sequence, a third foot member sequence, etc. It is further understood that the first foot member sequence, the second foot member sequence, the third foot member sequence, etc., can be used interchangeably.

In certain embodiments, the first foot member 32 can initiate varying stages of the ablation procedure depending on the first foot member sequence the user 11 selects and/or follows to actuate the first foot member 32. In other words, the first foot member 32 can initiate varying stages of the ablation procedure depending on the first foot member sequence selected by the user 11. In some embodiments, the first foot member sequence and the resulting initiation signal may be predetermined by the user 11 and may depend on certain preferences of user 11 and/or any other ablation parameters. As used herein, “predetermined” can include the user 11 selecting and programming the foot control assembly 26. In other embodiments, the first foot member sequence and the resulting initiation signal may be preprogrammed. As used herein, “preprogrammed” can mean preset and/or programmed as part of the foot member assembly 26.

In certain embodiments, the first foot member sequence the user 11 selects and/or follows to actuate the first foot member 32 can determine which stage of the ablation procedure will be initiated. As one non-exclusive example, when the first foot member 32 has been actuated a first time, the first foot member 32 can send the first initiation signal to the controller 28 to initiate the inflation stage. In the event the first foot member 32 has been actuated a plurality of times, i.e., a second time, the first foot member 32 can send the second initiation signal to the controller 28 to initiate the ablation stage. Further, in the event the first foot member 32 is actuated a plurality of times, i.e., a third time, the first foot member 32 can send the third initiation signal to the controller 28 to initiate the calculation and/or measurement of the time to isolation.

In another non-exclusive example, the length of time the user 11 depresses and holds down the first foot member 32 can determine which stage of the ablation procedure will be initiated. For example, if the user 11 depresses and holds down the first foot member 32 for half a second and releases, the inflation stage can be initiated. If the user 11 depresses and holds down the first foot member 32 for two seconds, the ablation stage can be initiated.

The method and/or manner in which the user 11 actuates the first foot member 32 can vary. In one embodiment, the first foot member 32 can include a foot pedal wherein certain stages of the ablation procedure can be initiated by the controller 28 depending on the first foot member sequence the user 11 selects and/or follows to depress the foot pedal. In another embodiment, the first foot member 32 can include a single button wherein stages of the ablation procedure can be initiated by the controller 28 depending on the first foot member sequence the user 11 selects and/or follows to depress the button. In yet another embodiment, the first foot member 32 can include a plurality of buttons, with each button corresponding to one of the stages of the ablation procedure, such that alternatingly depressing each of the buttons selectively causes the controller 28 to initiate one of the stages of the ablation procedure. In still another embodiment, the first foot member 32 can include a switch that can be selectively and/or manually moved or slid to enable the user 11 to cause the controller 28 to initiate one of the stages of the ablation procedure. Alternatively, the first foot member 32 can have any other suitable design that enables the user 11 to selectively and/or manually actuate the first foot member 32 to cause the controller 28 to initiate varying stages of the ablation procedure.

In various embodiments, the second foot member 34 can also be selectively and/or manually actuated by the user 11 to send a plurality of termination signals to terminate one or more stages of the ablation procedure. As one non-exclusive example, the second foot member 34 can be selectively and/or manually actuated by the user 11 to send a first termination signal to the controller 28. In this embodiment, while specific reference is made herein to sending the first termination signal, it is recognized that the second foot member 34 can send one or more termination signals, i.e., the first termination signal, a second termination signal, a third termination signal, etc. to terminate certain stages of the ablation procedure, which can be collectively referred to herein as a “termination signal.” It is further understood, that the first termination signal, the second termination signal, the third termination signal, etc. can be used interchangeably. Once actuated, the second foot member 34 can send the first termination signal to the controller 28. In various embodiments, the controller 28 can then process the first termination signal to terminate certain stages of the ablation procedure. Additionally, the second foot member 34 can have any suitable design so as to enable the user 11 to selectively and/or manually actuate the second foot member 34.

In certain embodiments, the second foot member 34 can terminate certain stages of the ablation procedure depending on the second foot member sequence the user 11 selects and/or follows to actuate the second foot member 34. More specifically, the function or operation of the second foot member 34 can depend on how the user 11 actuates the second foot member 34. In other words, the second foot member 34 can terminate certain stages of the ablation procedure depending on the second foot member sequence selected by the user 11 and/or preprogrammed as part of the foot member assembly 26. As referred to herein, the term “second foot member sequence” can include the method or manner in which the second foot member 34 is actuated, i.e., a number of times, an order, an arrangement, a series, a length of time, etc. and/or any combination thereof.

More specifically, in one non-exclusive example, in the event the second foot member 34 has been actuated a first time during the inflation stage, the second foot member 34 can send the first termination signal to the controller 28 to terminate the inflation stage. Alternatively, when the second foot member 34 has been actuated a first time during the ablation stage, the second foot member 34 can send the second termination signal to the controller 28 to terminate and/or stop the ablation stage. In some embodiments, when the second foot member 34 has been actuated a first time during the ablation stage, the thawing stage may also be initiated. The thawing stage can be initiated at any time at or after the ablation stage has been terminated or stopped, i.e., substantially simultaneously with the termination of the ablation stage, for example. In the event the second foot member 34 is actuated a plurality of times, i.e., a second time after the ablation stage has initiated and/or a first time during the thawing stage, the second foot member 34 can send a third termination signal to the controller 28 to terminate the ablation stage and/or the thawing stage. In various embodiments, should the inflation stage, the ablation stage and/or the thawing stage be terminated or stopped, the catheter system 10 may return to an idle position, at which time the controller 28 can reset the foot control assembly 26. In other words, the first foot member sequence of the first foot member 32 and/or the second foot member sequence of the second foot member 34 selected and/or followed by the user 11 is reset or recalibrated.

The method and/or manner in which the user 11 actuates the second foot member 34 can vary. In certain embodiments, the second foot member 34 can include a foot pedal, wherein certain stages of the ablation procedure can be terminated by the controller 28 depending on the second foot member sequence selected and/or followed by the user 11 to depress the foot pedal. In other embodiments, the second foot member 34 can include a single button wherein certain stages of the ablation procedure can be terminated by the controller 28 depending on the second foot member sequence selected and/or followed by the user 11 to depress the button. In yet other embodiments, the second foot member 34 can include a plurality of buttons, with each button corresponding to one of the stages of the ablation procedure, such that alternatingly depressing each of the buttons selectively causes the controller 28 to terminate one of the stages of the ablation procedure. In still other embodiments, the second foot member 34 can include a switch that can be selectively and/or manually moved or slid to enable the user 11 to cause the controller 28 to terminate one of the stages of the ablation procedure. Alternatively, the second foot member 34 can have any other suitable design that enables the user 11 to selectively and/or manually actuate the second foot member 34 to cause the controller 28 to terminate certain stages of the ablation procedure.

In one non-exclusive embodiment, the foot control assembly 26 may include only the first foot member 32. In this embodiment, the first foot member 32 can initiate and terminate certain stages of the ablation procedure depending on the first foot member sequence the user 11 selects and/or follows to actuate the first foot member 32. In other words, the first foot member 32 can initiate and terminate certain stages of the ablation procedure depending on the first foot member sequence selected by the user 11 and/or preprogrammed as part of the foot member assembly 26. For instance, when the first foot member 32 has been actuated a first time, the first foot member 32 can send the first initiation signal to the controller 28 to initiate the inflation stage. In the event the first foot member 32 has been actuated a plurality of times, the first foot member 32 can send either the second initiation signal to the controller 28 to initiate the ablation stage and/or the third initiation signal to the controller 28 to initiate the calculation and/or measurement of the time to isolation. Additionally, in the event the first foot member 32 has been actuated a plurality of times, the first foot member can send either the first termination signal to the controller 28 to terminate the inflation stage, the second termination signal to the controller 28 to terminate the ablation stage and/or the third termination signal to the controller 28 to terminate the thawing stage.

In another non-exclusive embodiment, the first foot member 32 and/or the second foot member 34 can allow the user 11 to control a flow rate of the cryogenic fluid 27 to and/or from the balloon catheter 18. In other words, the first foot member 32 and/or the second foot member 34 can control the cryogenic fluid 27 that is released to the balloon catheter 18 during the ablation procedure, which may adjust (i.e., increase or decrease) and/or maintain an inflatable balloon size, a temperature and/or a pressure within the inflatable balloon of the balloon catheter 18. As used herein, the term “control” can include to initiate, increase or decrease. More specifically, the user 11 can depress and hold down the first foot member 32 and/or second foot member 34 in order to achieve or reach, a desired flow rate, temperature and/or pressure. Further, the user 11 can depress and hold down the first foot member 32 and/or second foot member 34 in order to achieve or reach the desired inflatable balloon size. While in this embodiment, the method of depressing is described, it is understood that the first foot member 32 and/or second foot member 34 may be moved, slid, etc. and held. Once the desired flow rate, inflatable balloon size, temperature and/or pressure is achieved, the user 11 can release the first foot member 32 and/or the second foot member 34. As the first foot member 32 and/or the second foot member 34 is released, the desired flow rate, inflatable balloon size, temperature and/or pressure may be maintained. As used herein, the term “maintain” means to keep, sustain, preserve, etc. substantially the same flow rate, inflatable balloon size, temperature and/or pressure as at the time the first foot member 32 and/or the second foot member 34 was released.

In one embodiment, the first foot member 32 can be depressed and held down a first time to send a first depression signal to the controller 28 to control, i.e., initiate and/or increase, the flow of cryogenic fluid 27 until the desired flow rate, inflatable balloon size, temperature and/or pressure for the initiation stage is achieved or reached. Once the desired flow rate, inflatable balloon size, temperature and/or pressure for the initiation stage is achieved, the user 11 can release the first foot member 32. As the first foot member 32 is released, the first foot member 32 can send a first release signal to the controller 28 to maintain the desired flow rate, inflatable balloon size, temperature and/or pressure for the inflation stage. Further, the first foot member 32 can be depressed and held down a second time to send the first depression signal to the controller 28 to control, i.e., increase, the flow rate of the cryogenic fluid 27 until the desired flow rate, inflatable balloon size, temperature and/or pressure for the ablation stage is achieved. Once the desired flow rate, inflatable balloon size, temperature and/or pressure for the ablation stage is achieved, the user 11 can release the first foot member 32. As the first foot member 32 is released, the first foot member 32 can send the first release signal to the controller 28 to maintain the desired flow rate, inflatable balloon size, temperature and/or pressure for the ablation stage. Still further, the second foot member 34 can be depressed and held down at any point during the ablation procedure to send a second depression signal to the controller 28 to control, i.e., decrease, the flow rate of the cryogenic fluid 27. For example, the second foot member 34 can be depressed and held down by the user 11 until the desired flow rate, inflatable balloon size, temperature and/or pressure for thawing stage has been achieved. Once the desired flow rate, inflatable balloon size, temperature and/or pressure for the thawing stage is achieved, the user 11 can release the second foot member 34 to send a second release signal to the controller 28 to maintain the desired flow rate, inflatable balloon size, temperature and/or pressure for the thawing stage.

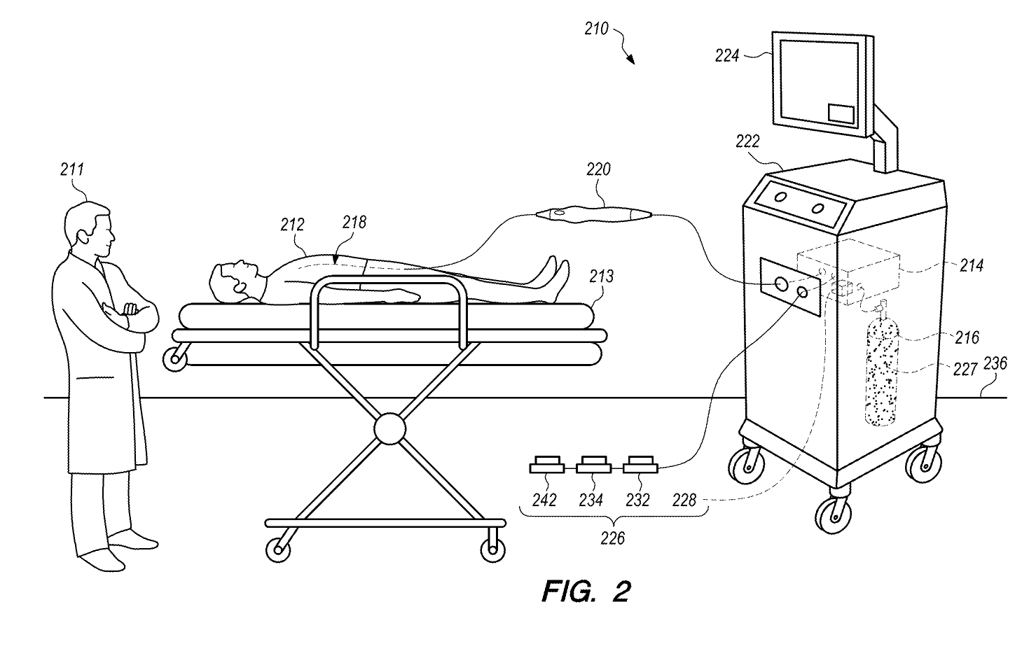

FIG. 2 is a schematic side view of the user 211, the patient 212 and another embodiment of the catheter system 210. In the embodiment illustrated in FIG. 2, the catheter system 210 includes the control system 214, the fluid source 216, the balloon catheter 218, the handle assembly 220, the control console 222, the GUI 224 and the foot control assembly 226. However, in the embodiment illustrated in FIG. 2, the foot control assembly 226 includes the controller 228 and the plurality of foot members, i.e., the first foot member 232, the second foot member 234 and a third foot member 242.

In this embodiment, the foot control assembly 226 includes several structures which are coupled and/or connected to each other and the controller 228. Alternatively, the first foot member 232, the second foot member 234 and the third foot member 242 can be separately coupled and/or connected to the controller 228. The controller 228, the first foot member 232, the second foot member 234 and the third foot member 242 can be coupled and/or connected via any suitable manner.

In the embodiment illustrated in FIG. 2, the controller 228 can be integrated and/or included as part of the control system 214. In other embodiments, the controller 228 can be separate and/or apart from the control system 214, and integrated and/or included as part of the control console 222, for example. Additionally, and/or alternatively, the controller 228 can be integrated and/or included as part of any other suitable structure in the catheter system 210.

Additionally, in FIG. 2, the user 211, the gurney 213, the control console 222 and the foot control assembly 226 are positioned, situated and/or placed on or near the support surface 236.

In certain embodiments, the third foot member 242 can be selectively and/or manually actuated by the user 211 to send a timer signal or a plurality of timer signals. As one non-exclusive example, the third foot member 242 can be selectively and/or manually actuated by the user 211 to send a first timer signal to the controller 228. In this embodiment, while specific reference is made herein to sending the first timer signal, it is recognized that the third foot member 242 can send one or more timer signals, i.e., the first timer signal, a second timer signal, etc. to initiate and/or terminate timers, which can be collectively referred to herein as a “timer signal.” It is further understood that the first timer signal, the second timer signal, etc., can be used interchangeably.

Once actuated, the third foot member 242 can send one or more timer signals to the controller 228. In various embodiments, the controller 228 can then process each timer signal to initiate and/or terminate certain timers. As used herein, “timers” can include the monitoring and/or recording of time for any suitable function of the catheter system 210. In one embodiment, the timer can be configured to monitor elapsed time during the ablation procedure until the time to isolation is achieved. In another embodiment, the timer can be configured to monitor elapsed time from the beginning of the ablation procedure to when targeted tissue is effectively isolated and non-conducting, i.e., at the time to isolation. In various embodiments, the third foot member 242 can be substantially similar in design and/or configuration to the first foot member 232 and the second foot member 234. Alternatively, the third foot member 242 can have any other suitable design so as to enable the user 211 to selectively and/or manually actuate the third foot member 242.

In some non-exclusive embodiments, the third foot member 242 can be configured to specifically provide the user 211 with the means to selectively and/or manually actuate the third foot member 242 to cause the controller 228 to initiate and/or terminate timers during varying stages of the ablation procedure. In certain embodiments, the third foot member 242 can initiate and/or terminate timers during varying stages of the ablation procedure depending on the third foot member sequence the user 211 selects and/or follows to actuate the third foot member 242. In other words, the third foot member 242 can initiate and/or terminate timers during varying stages of the ablation procedure depending on the third foot member sequence selected by the user 211. For example, in certain embodiments, the third foot member 242 can initiate or terminate certain timers depending on the number of times the user 211 actuates, i.e., depresses, moves, slides, etc., the third foot member 242. Alternatively, the third foot member 242 can initiate or terminate timers depending on the length of time the user 211 holds down the third foot member 242.

The method and/or manner in which the user 211 actuates the third foot member 242 can vary. In certain embodiments, the third foot member 242 can include a foot pedal wherein timers during varying stages of the ablation procedure can be initiated and/or terminated by the controller 228 depending on the third foot member sequence selected and/or followed by the user 211 to depress the foot pedal. More specifically, in one non-exclusive embodiment, when the third foot member 242 has been actuated and/or depressed a first time, the third foot member 242 can send the first timer signal to the controller 228 to initiate the timer. In the event the third foot member 242 has been actuated and/or depressed a plurality of times, i.e., a second time, the third foot member 242 can send the second timer signal to the controller 228 to terminate the timer. Additionally, and/or alternatively, the third foot member 242 can have any other suitable design that effectively enables the user 211 to selectively and/or manually actuate the third foot member 242 to cause the controller 228 to initiate and/or terminate timers.

In one non-exclusive embodiment, the third foot member 242 can function to activate and/or deactivate the foot control assembly 226. More specifically, in certain embodiments, while the foot control assembly 226 is in the idle position the user 211 can actuate the third foot member 242 to send a deactivation signal to the controller 228 to deactivate the foot control assembly 226. Additionally, the user 211 can actuate the third foot member 242 to send an activation signal to the controller 228 to activate or reactivate the foot control assembly 226. For example, the user 211 can depress the third foot member 242 a first time for a certain period of time or number of times, i.e., x amount of seconds or times, to send the deactivation signal to the controller 228 to deactivate the foot control assembly 226. This may have the effect of relatively minimizing any accidental initiation of the inflation stage and/or ablation stage by the user 211. The user 211 can also depress the third foot member 242 the plurality of times, i.e., second time, to send the activation signal to the controller 228 to activate or reactivate the foot control assembly 226.

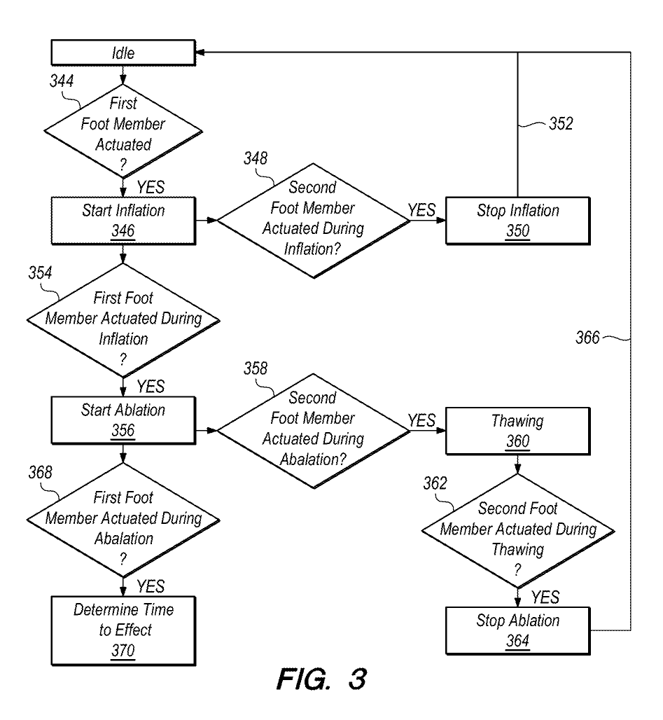

FIG. 3 is a flowchart illustrating one embodiment of a method for operating the foot control assembly 326. It is appreciated that the order of the steps illustrated and described in FIG. 3 is not necessarily indicative of how the foot control assembly 326 operates chronologically, as one or more of the steps can be combined, reordered, repeated and/or performed simultaneously without deviating from the intended breadth and scope of the foot control assembly 326 and method. It further is recognized that the flowchart shown in FIG. 3 is merely one representative example of how the foot control assembly 326 can be utilized within the catheter system 310 and is not intended to be limiting in any manner.

At step 344, a determination is made whether the first foot member is actuated. The first foot member is actuated when the first foot member is depressed, moved, slid, etc. by the user.

At step 346, in the event the first foot member has been actuated the first time, the first foot member sends the first initiation signal to the controller to initiate the inflation stage, i.e., to “Start Inflation”.

At step 348, a determination is made whether the second foot member is actuated. The second foot member is actuated when the second foot member is depressed, moved, slid, etc. by the user during the inflation stage or the first time.

At step 350, in the event the second foot member is actuated during the inflation stage or the first time, the second foot member sends the first termination signal to the controller to terminate or stop the inflation stage, i.e., to “Stop Inflation”.

At step 352, in the event the inflation stage is terminated or stopped, the catheter system may return to the idle position, i.e., “Idle”, at which time the controller can reset or recalibrate the foot control assembly.

At step 354, a determination is made whether the first foot member has been actuated during the inflation stage or the second time.

At step 356, in the event the first foot member is actuated during the inflation stage or the second time, the first foot member sends the second initiation signal to the controller to initiate the ablation stage, i.e., to “Start Ablation”.

At step 358, a determination is made whether the second foot member has been actuated during the ablation stage or the first time after the ablation stage has been initiated.

At step 360, in the event the second foot member is actuated during the ablation stage or the first time after the ablation stage has initiated, the second foot member sends the second termination signal to the controller to terminate the ablation stage. In some embodiments, the second termination signal may also initiate the thawing stage. The second termination signal can initiate the thawing stage substantially at or after the time the ablation stage has been terminated or stopped.

At step 362, a determination is made whether the second foot member has been actuated the first time during the thawing stage or the second time after the ablation stage has initiated.

At step 364, in the event the second foot member is actuated the first time during the thawing stage and/or the second time after the ablation stage has initiated, the second foot member sends the third termination signal to the controller to terminate or stop the ablation stage and/or the thawing stage, i.e., to “Stop Ablation”.

At step 366, in the event the ablation stage and/or the thawing stage are terminated or stopped, the catheter system may return to the idle position, i.e., “Idle”, at which time the controller can reset or recalibrate the foot control assembly.

At step 368, a determination is made whether the first foot member has been actuated during the ablation stage or the third time.

At step 370, in event the first foot member is actuated during the ablation stage or the third time, the first foot member sends the third initiation signal to the controller to initiate the calculation and/or measurement of the time to isolation or time to effect.

It is understood that although a number of different embodiments of the catheter system and/or the foot control assembly have been illustrated and described herein, one or more features of any one embodiment can be combined with one or more features of one or more of the other embodiments, provided that such combination satisfies the intent of the present disclosure.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

I claim:

1. An ablation system for use in performing an ablation procedure, the ablation system comprising:
an ablation catheter having an ablation element configured to be advanced within a body of a patient and to deliver ablative energy to target tissue of the patient;
a control console operatively coupled to the ablation catheter and including an ablation energy supply; and
a controller operatively coupled to the control console and configured to control one or more stages of the ablation procedure; and
first and second foot members configured to be positioned remotely from the control console at a location where the first and second foot members can be manually operated by a foot of a user while the user is manipulating the ablation catheter within the patient, each of the first and second foot members being operatively coupled to the controller and configured to be selectively actuated by the user to selectively send one of: (i) at least one initiation signal to the controller to initiate a stage of the ablation procedure, and (ii) at least one termination signal to the controller to terminate a stage of the ablation procedure.

2. The ablation system of claim 1, wherein one of the first and second foot members is configured to be selectively actuated by the user to send the at least one initiation signal to the controller, and the other of the first and second foot members is configured to be selectively actuated by the user to send the at least one termination signal to the controller.

3. The ablation system of claim 1, wherein the ablation procedure includes an inflation stage, and wherein the at least one initiation signal initiates the inflation stage, and wherein the at least one termination signal terminates the inflation stage.

4. The ablation system of claim 1, wherein the ablation procedure includes an ablation stage, and wherein the at least one initiation signal initiates the ablation stage, and wherein the at least one termination signal terminates the ablation stage.

5. The ablation system of claim 1, wherein the ablation procedure includes a time to isolation, and wherein the at least one initiation signal initiates a calculation of the time to isolation.

6. The ablation system of claim 1, wherein the ablation procedure includes an ablation stage and a thawing stage, and wherein the at least one termination signal terminates the ablation stage and substantially simultaneously initiates the thawing stage.

7. The ablation system of claim 1, wherein the at least one initiation signal includes a timer signal to the controller to initiate a timer configured to monitor time elapsed during a stage of the ablation procedure.

8. The ablation system of claim 1, wherein one of the first and second foot members is configured to be selectively actuated by the user according to a first foot member sequence to send at least one of (i) a plurality of initiation signals to the controller to initiate at least one stage of the ablation procedure, and (ii) a plurality of termination signals to the controller to terminate at least one stage of the ablation procedure.

9. The ablation system of claim 1, wherein the controller and the first and second foot members are incorporated into a foot control assembly that is configured to be located remotely from the control console.

10. An ablation system for use in performing an ablation procedure, the ablation system comprising:
an ablation catheter having an ablation element configured to be advanced within a body of a patient and to deliver ablative energy to target tissue of the patient;
a control console operatively coupled to the ablation catheter and including an ablation energy supply; and
a foot control assembly operatively coupled to the control console and configured to be positioned remotely from the control console at a location where the foot control assembly can be manually operated by a foot of a user while the user is manipulating the ablation catheter within the patient, the foot control assembly comprising:
a controller configured to control one or more stages of the ablation procedure; and
a first foot member that is configured to be selectively actuated by the user according to a first foot member sequence to send at least one of (i) a plurality of initiation signals to the controller to initiate at least one stage of the ablation procedure, and (ii) a plurality of termination signals to the controller to terminate at least one stage of the ablation procedure.

11. The ablation system of claim 10, wherein the ablation procedure includes a plurality of stages including an inflation stage, an ablation stage and a thawing stage, and wherein the first foot member is configured to be selectively actuated by the user to send a first initiation signal to the controller to initiate one of the plurality of stages, and to be selectively actuated by the user to send a second initiation signal to the controller to initiate a different one of the plurality of stages.

12. The ablation system of claim 11, wherein the ablation procedure includes a time to isolation, and wherein the first foot member is configured to be selectively actuated by the user to send a third initiation signal to the controller to initiate a calculation of the time to isolation.

13. The ablation system of claim 10, wherein the ablation procedure includes a plurality of stages including an inflation stage, an ablation stage and a thawing stage, and wherein the first foot member is configured to be selectively actuated by the user to send a first termination signal to the controller to terminate one of the plurality of stages, and to be selectively actuated by the user to send a second termination signal to the controller to terminate a different one of the plurality of stages.

14. The ablation system of claim 10, wherein the first foot member is configured to be selectively actuated by the user to send a timer signal to the controller to initiate a timer configured to monitor an elapsed time during a stage of the ablation procedure.

15. The ablation system of claim 10, further comprising a second foot member that is configured to be selectively actuated by the user according to a second foot member sequence to send at least one of (i) the plurality of initiation signals to the controller to initiate at least one stage of the ablation procedure, and (ii) the plurality termination signals to the controller to terminate at least one stage of the ablation procedure.

16. A method for controlling at least one stage of an ablation procedure, the method comprising:
manipulating an ablation catheter so as to position an ablation element thereof at a location proximate target tissue of a patient; and
selectively actuating a first foot member according to a first foot member sequence to send at least one at least one of (i) a plurality of initiation signals to a controller to initiate at least one stage of the ablation procedure, and (ii) a plurality of termination signals to the controller to terminate at least one stage of the ablation procedure,
wherein the foot member is operatively coupled to the controller and is positioned at a location where the first foot member can be actuated by a foot of a user while the user is manipulating the ablation catheter within the patient.

17. The method of claim 16, wherein the ablation procedure includes a plurality of stages including an inflation stage, an ablation stage and a thawing stage, and wherein selectively actuating includes selectively actuating the first foot member to send a first initiation signal to the controller to initiate one of the plurality of stages, and selectively actuating the first foot member to send a second initiation signal to the controller to initiate a different one of the plurality of stages.

18. The method of claim 17, wherein selectively actuating includes selectively actuating the first foot member to send a third initiation signal to the controller to calculate a time to isolation.

19. The method of claim 17, wherein selectively actuating includes selectively actuating the first foot member to send a first termination signal to the controller to terminate the one of the plurality of stages, and selectively actuating the first foot member to send a second termination signal to the controller to terminate the different one of the plurality of stages.

20. The method of claim 17, further comprising selectively actuating a second foot member of the foot control assembly according to a second foot member sequence to send a timer signal to the controller to initiate a timer to monitor an elapsed time of a selected one of the plurality of stages.
